(19) United States
(12) Patent Application Publication
(10) Pub. No.: US 2022/0406451 A1
(43) Pub. Date: Dec. 22, 2022

(54) Integrative Multi-Modal Computing For Personal Health Navigation

(71) Applicants: Zepp, Inc., Cupertino, CA (US); Anhui Huami Health Technology Co., Ltd., Hefei (CN)

(72) Inventors: Nitish Nag, Cupertino, CA (US); Hyungik Oh, Cupertino, CA (US); Mengfan Tang, Cupertino, CA (US); Ramesh Jain, Cupertino, CA (US)

(21) Appl. No.: 17/830,571

(22) Filed: Jun. 2, 2022

Related U.S. Application Data
(60) Provisional application No. 63/212,542, filed on Jun. 18, 2021.

(51) Int. Cl. G16H 40/63

(52) U.S. Cl. CPC G16H 40/63

(57) ABSTRACT

Method and apparatus for personal health navigation includes determining, by a processor, a personal health state space comprising a set of connected biological states for an individual; determining, for the individual, a region of interest within the personal health state space, in which the region of interest is defined by a goal state relative to a current state of the individual, the goal state associated with a personal health goal of the individual; determining a route comprising the current state, the goal state, and an intermediate state within the personal health state space, the intermediate state closer in distance to the goal state than the current state; and providing, to the individual, a health instruction indicative of a recommended action based on a connection between the current state and the intermediate state, in which the recommended action is selected to guide the individual to transition to the intermediate state.

### CROSS-REFERENCE TO RELATED APPLICATION(S)

This application claims priority to and the benefit of U.S. Provisional Application Patent Ser. No. 63/212,542, filed Jun. 18, 2021, the entire disclosure of which is hereby incorporated by reference.

### TECHNICAL FIELD

This application relates to wearable computing, in particular to integrative multi-modal computing for personal health navigation.

### BACKGROUND

With modern technologies, we have the ability to sense and compute upon health-related data ubiquitously and continuously, and apply this information towards improved health.

Current research in health recommender systems generally take the route of complete automation or melding human expertise with computing aid for decisions making. Automated systems for health recommendation are quite limited in their function, mostly being used to maintain simple homeostatic control, such as monitoring glucose with an insulin pump. Human experts may be utilized for recommending inputs to an individual's health state in person or through telecommunication and decision aid systems. These forms of guidance can be synchronous in real-time, or they can be asynchronous through various forms of communication and multi-media.

### SUMMARY

The disclosure relates in general to wearable computing, in particular to integrative multi-modal computing for personal health navigation. Aspects of this disclosure include, for example, a method, apparatus and non-transitory computer readable medium for personal health navigation.

One aspect of the disclosed implementations is a method for personal health navigation. The method includes determining, by a processor, a personal health state space comprising a set of connected biological states for an individual; determining, for the individual and by the processor, a region of interest within the personal health state space, wherein the region of interest is defined by a goal state relative to a current state of the individual, the goal state associated with a personal health goal of the individual; determining, by the processor, a route comprising the current state, the goal state, and an intermediate state within the personal health state space, the intermediate state closer in distance to the goal state than the current state; and providing, to the individual and by the processor, a health instruction indicative of a recommended action based on a connection between the current state and the intermediate state, wherein the recommended action is selected to guide the individual to transition to the intermediate state.

In some implementations, the method can further include, for example, one or more of the following features: upon determining that the individual has completed the recommended action, the current state is transitioned into the intermediate state; upon determining that the individual has taken a different action wherein the current state transitions into another state different from the intermediate state, recalculating the route for the individual based on the another state and the goal state; the region of interest is semantically labeled with domain knowledge associated with the personal health goal; receiving, from the individual, input indicative of the personal health goal; and decomposing, by the processor, the personal health goal into sub-goals represented as nodes in the region of interest; determining, by the processor, a state transition network comprising edges, each edge representative of a transition from a first state to a second state based on a personal model associated with the individual; determining, by the processor, the route comprising the current state, the goal state, and the intermediate state further comprises: determining, for the individual associated with the personal health goal, the route leading from the current state to the goal state within the state transition network, wherein the route comprises an optimal subset of states between the current state and the goal state, and corresponding edges; the health instruction indicative of the recommended action comprises one of: a lifestyle event or a medical event; the health instruction indicative of the recommended action comprises one of: a lifestyle event or a medical event; or, the personal health state space comprises a subset of possible biological states for the individual within a multi-dimensional general health state space, the subset of possible biological states determined based on characteristics specific to the individual.

Another aspect of the disclosed implementations is an apparatus for personal health navigation. The apparatus includes a non-transitory memory; and a processor configured to execute instructions stored in the non-transitory memory to: determine a personal health state space comprising a set of connected biological states for an individual; determine, for the individual, a region of interest within the personal health state space, wherein the region of interest is defined by a goal state relative to a current state of the individual, the goal state associated with a personal health goal of the individual; determine, by the processor, a route comprising the current state, the goal state, and an intermediate state within the personal health state space, the intermediate state closer in distance to the goal state than the current state; and provide, to the individual and by the processor, a health instruction indicative of a recommended action based on a connection between the current state and the intermediate state, wherein the recommended action is selected to guide the individual to transition to the intermediate state.

In some implementations, the apparatus can further include, for example, one or more of the following features: upon determining that the individual has completed the recommended action, the current state is transitioned into the intermediate state; upon determining that the individual has taken a different action wherein the current state is transitioned into another state different from the intermediate state, recalculating the route for the individual based on the another state and the goal state; the region of interest is semantically labeled with domain knowledge associated with the personal health goal; the instructions further comprise instructions to: receive, from the individual, input indicative of the personal health goal; and decompose the personal health goal into sub-goals represented as nodes in the region of interest; the instructions further comprise instructions to: determine a state transition network comprising edges, each edge representative of a transition from a first state to a second state based on a personal model associated with the individual; the instructions to determine the route comprising the current state, the goal state, and the intermediate state within the region of interest further comprise instructions to: determine, for the individual associated with the personal health goal, the route leading from the current state to the goal state within the state transition network, wherein the route comprises an optimal subset of states between the current state and the goal state, and corresponding edges; the health instruction indicative of the recommended action comprises one of: a lifestyle event or a medical event, and wherein the current state is estimated based on physiological measurements of the individual; or, the personal health state space comprises a subset of possible biological states for the individual within a multi-dimensional general health state space, the subset of possible biological states determined based on characteristics specific to the individual.

Another aspect of the disclosed implementations is a non-transitory computer-readable storage medium configured to store computer programs for personal health navigation. The computer programs include instructions executable by a processor to: determine a personal health state space comprising a set of connected biological states for an individual; determine, for the individual, a region of interest within the personal health state space, wherein the region of interest is defined by a goal state relative to a current state of the individual, the goal state associated with a personal health goal of the individual; determine, by the processor, a route comprising the current state, the goal state, and an intermediate state within the personal health state space, the intermediate state closer in distance to the goal state than the current state; and provide, to the individual and by the processor, a health instruction indicative of a recommended action based on a connection between the current state and the intermediate state, wherein the recommended action is selected to guide the individual to transition to the intermediate state.

### BRIEF DESCRIPTION OF DRAWINGS

FIG. 1 is a block diagram illustrating an example of personal health navigation (PHN) according to an implementation.

FIG. 2 is a block diagram of an example of a computing device that can be used to implement the functionalities of PHN according to implementations of this disclosure.

FIG. 3 is a diagram showing an example process of PHN according to implementations of this disclosure.

FIG. 4 is a flow diagram illustrating an example system framework for PHN.

FIG. 5A is an example diagram illustrating PHN in a cardiovascular health setting.

FIG. 5B is an example diagram illustrating multiple user trends for PHN according to experimental data collected from the cardiovascular health setting.

FIG. 5C shows an example flow diagram of a daily exercise guidance algorithm for PHN in the cardiovascular health setting.

### DETAILED DESCRIPTION

As mobile health care market size keeping growing, devices and systems using wearable technologies to aid fitness or health assessments have become widely used. Wearable devices, such as smart watches and fitness bands, have been used for monitoring health status and tracking fitness of individuals. Wearable devices can be used for a variety of applications, such as, for example, step counting, activity tracking or calorie-burn estimation. Current wearable devices mainly show data-streams back to the user, without interpretation or actionable information. This makes the device less useful and relevant for people to live a healthy lifestyle.

Good health provides the foundation by which people can live happy and productive lives. It is well understood that an individual's health trajectory is influenced by choices made in each moment, such as from lifestyle or medical decisions. With the advent of modern sensing technologies, individuals have more data and information about themselves than any other time in history. It remains challenging to transform this collected data to real-world improvements in individual health. Furthermore, delivering better health quality to people without excessive cost is also key to allow societal resources to go towards progress in other domains.

In order to make this abundance of data actionable and relevant to keep the individuals healthy, methods, apparatuses and systems for Personal Health Navigation (PHN) are proposed. PHN takes the individuals towards their personal health goals by, for example, digesting multi-modal data streams, estimating a current health status, computing the best route(s) through intermediate states given personal models, and providing guidance on actionable inputs that take the individuals towards their personal health goals, among other things.

According to implementations of this disclosure, wearable data measured from the individual can be used to guide the individual towards a desired healthy lifestyle state in a personalized, adaptive, and contextual manner for the individual to exercise in a way that can improve his or her fitness level (e.g., cardiorespiratory fitness).

According to implementations of this disclosure, the capabilities of cybernetic control and long-term intelligent planning can be built into PHN.

Example implementations of the present disclosure will be described below with reference to the accompanying drawings. The same numbers across the drawings set forth in the following description represent the same or similar elements, unless differently expressed. The implementations set forth in the following description do not represent all implementations or embodiments consistent with the present disclosure; on the contrary, they are only examples of apparatuses and methods in accordance with some aspects of this disclosure as detailed in the claims.

It should be noted that the applications and implementations of this disclosure are not limited to the examples, and alternations, variations, or modifications of the implementations of this disclosure can be achieved for any computation environment.

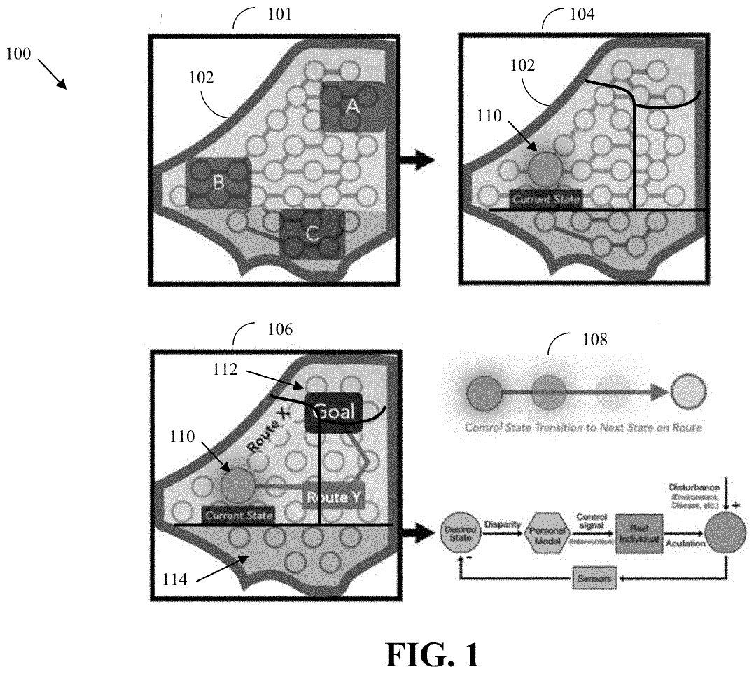

FIG. 1 is a block diagram 100 illustrating an example of personal health navigation (PHN) according to an implementation. In FIG. 1, the concepts of personal health navigation are shown in an illustrative example. In this example, PHN guides an individual (also referred to herein as “user”) toward that individual's personal health goal(s). A personal health goal can be defined computationally in terms of a region-of-interest (ROI) within a multi-dimensional space, where each dimension of the multi-dimensional space represents a different component of personal health. The different components of personal health can be defined through, for example, biomedical knowledge. These dimensions are converted into discrete biological states (also referred to as “nodes”, “health states” or “states”) as shown in FIG. 1, which form a General Health State Space (GHSS) 101 that serves as a base map for PHN. The states are then connected via input knowledge, which can be knowledge-driven at cold-start and then iteratively improved with data-driven analysis.

For a particular user such as the individual described above, there is only a subset of the GHSS 101 that the individual can access due to his or her biological uniqueness. This subset is referred to as a Personal Health State Space (PHSS) 102, which includes all possible states for the individual given the personal situation. As seen in FIG. 1, the PHSS 102 is shown with thick lines as boundaries inside the GHSS 101. The PHSS 102 can be labeled with different ROIs, such as, for example, a ROI 112 and a ROI 114 as shown in FIG. 1, from which the individual may select one as a goal. The PHSS 102 also includes edges between states, where the edges represent the knowledge of the individual's actions (also referred to herein as “inputs”) to perform a state transition.

Once the PHSS 102 is determined, a Health State Estimation (HSE) 104 is used to determine a current state 110 of the individual on the PHSS 102. The current state 110 indicates a current status of health for the individual. Once the individual provides a goal, as shown in the example of a Route Planning and Selection 106 of FIG. 1, the goal is mapped to the ROI 112, and the individual is provided with various routes from the current state 110 to the goal state (represented by the ROI 112 in this example) to select from. Upon selection of a route, a system implementing this example transitions to a Cybernetic Control 108, where control mechanisms are implemented to ensure a smooth transition to the next neighboring state along the selected route. The individual is guided through the Cybernetic Control 108 to perform inputs (actions) to arrive at the next neighboring state on the selected route. Inputs/actions carried out by the individual, including the ones advised by the system or ones that are not or both, are measured and fed into a new estimation of the current state 110 of the individual, and controlled using the Cybernetic Control 108 to stay on track. The updated current state 110 is then used to update the next proposed action. A cycle including the Route Planning and Selection 106 (for re-planning when the current state 110 is updated) and the Cybernetic Control 108 is performed repeatedly, which is used to produce movement of the individual's health state closer to the goal state, until the individual reaches (and in some cases, maintains) the goal state (in this example the ROI 112) as a destination. Upon reaching the destination, the system can continue to ensure that deviation from the goal state is minimized. More details, examples and implementations are described below in connection with the remaining figures.

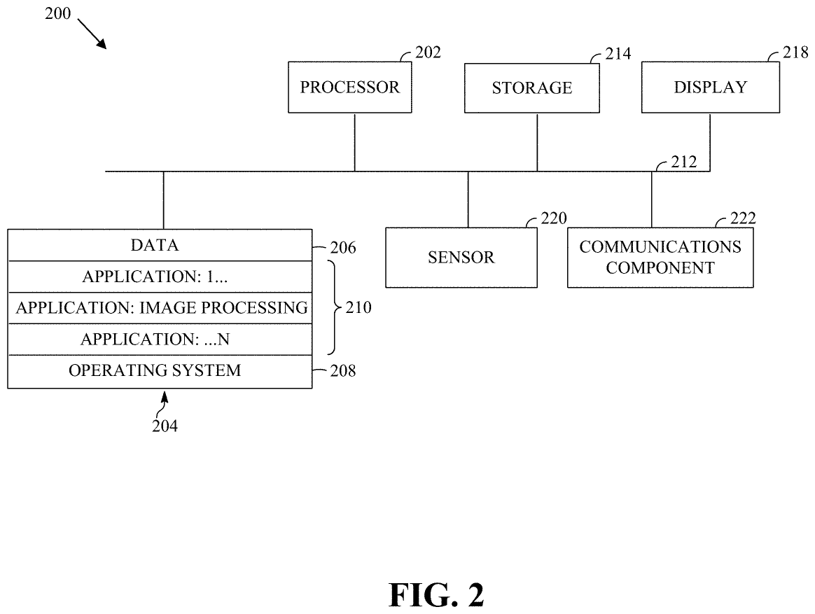

FIG. 2 is a block diagram of an example of a computing device 200 that can be used to implement the functionalities of personal health navigation according to implementations of this disclosure. The computing device 200 can be in the form of a computing system including multiple computing devices, or in the form of a single computing device, for example, a mobile phone, a tablet computer, a laptop computer, a notebook computer, a desktop computer, a wearable device, a smart scale or the like.

A CPU 202 in the computing device 200 can be a central processing unit. Alternatively, the CPU 202 can be any other type of device, or multiple devices, capable of manipulating or processing information now-existing or hereafter developed. Although the disclosed implementations can be practiced with a single processor as shown, e.g., the CPU 202, advantages in speed and efficiency can be achieved using more than one processor.

A memory 204 in the computing device 200 can be a read-only memory (ROM) device or a random access memory (RAM) device in an implementation. Any other suitable type of storage device can be used as the memory 204. The memory 204 can include code and data 206 that is accessed by the CPU 202 using a bus 212. The memory 204 can further include an operating system 208 and application programs 210, the application programs 210 including at least one program that permits the CPU 202 to perform the methods described here. For example, the application programs 210 can include applications 1 through N, which further include an application incorporating some or all of the personal health navigation features. The computing device 200 can also include a secondary storage 214, which can, for example, be a memory card used with a computing device 200 that is mobile.

The computing device 200 can also include one or more output devices, such as a display 218. The display 218 can be, in one example, a touch sensitive display that combines a display with a touch sensitive element that is operable to sense touch inputs. The display 218 can be coupled to the CPU 202 via the bus 212. Other output devices that permit a user to program or otherwise use the computing device 200 can be provided in addition to or as an alternative to the display 218. When the output device is or includes a display, the display can be implemented in various ways, including by a liquid crystal display (LCD), a cathode-ray tube (CRT) display or light emitting diode (LED) display, such as an organic LED (OLED) display.

The computing device 200 can include or be in communication with one or more sensors 220 that can measure one or more types of wearable data of a user. The sensors can include, for example, camera, microphone, accelerometer, gyroscope, an inertia measurement unit (IMU) sensor, a magnetometer, PPG or ECG heartrate sensor, EKG sensor, light sensor, SpO2 sensor, GPS, camera, Dot Projector, temperature sensor, humidity sensor, barometer etc. The sensors can be located, for example, in a smart earbud, on a watch, wristband, or mobile phone, on a smart scale, at a laptop or TV, a home IOT device, in a connected car or the like.

The computing device 200 can include or be in communication with a communications component 222, which can be a hardware or software component configured to communicate data to one or more external devices, such as another computing device or a wearable device, for example. The communication component can operate over wired or wireless communication connections, such as, for example, a wireless network connection, a Bluetooth connection, an infrared connection, an NFC connection, a cellular network connection, a radio frequency connection, or any combination thereof. In some implementations, the communications component comprises an active communication interface, for example, a modem, transceiver, or the like. In some implementations, the communications component comprises a passive communication interface, for example, a quick response (QR) code, Bluetooth identifier, radio-frequency identification (RFID) tag, a near-field communication (NFC) tag, or the like. In some implementations, the communication component can use sound signals as input and output, such as, for example, an ultrasonic signal or a sound signal via an audio jack. Implementations of the communications component can include a single component, one of each of the foregoing types of components, or any combination of the foregoing components.

Although FIG. 2 depicts the CPU 202 and the memory 204 in the computing device 200, other configurations can be utilized. The operations of the CPU 202 can be distributed across multiple machines (each machine having one or more of processors) that can be coupled directly or across a local area or other network. The memory 204 can be distributed across multiple machines such as a network-based memory or memory in multiple machines performing the operations of the computing device 200. Although depicted here as a single bus, the bus 212 of the computing device 200 can be composed of multiple buses. Further, the secondary storage 214 can be directly coupled to the other components of the computing device 200 or can be accessed via a network and can comprise a single integrated unit such as a memory card or multiple units such as multiple memory cards. The computing device 200 can thus be implemented in a wide variety of configurations.

Computing device 200 is shown as an example in FIG. 2, but it is not limited to any specific type or any specific quantity in the system disclosed herein. Computing device 200 can be implemented by any configuration of one or more computers, such as a microcomputer, a mainframe computer, a super computer, a general-purpose computer, a special-purpose/dedicated computer, an integrated computer, a database computer, a remote server computer, a personal computer, a laptop computer, a tablet computer, a cell phone, a personal data assistant (PDA), a wearable computing device, e.g., a smart watch, or a computing service provided by a computing service provider, e.g., a website, or a cloud service provider. In some implementations, certain operations described herein can be performed by a computer (e.g., a server computer) in the form of multiple groups of computers that are at different geographic locations and can or cannot communicate with one another by way of, such as, a network. While certain operations can be shared by multiple computers, in some implementations, different computers can be assigned with different operations.

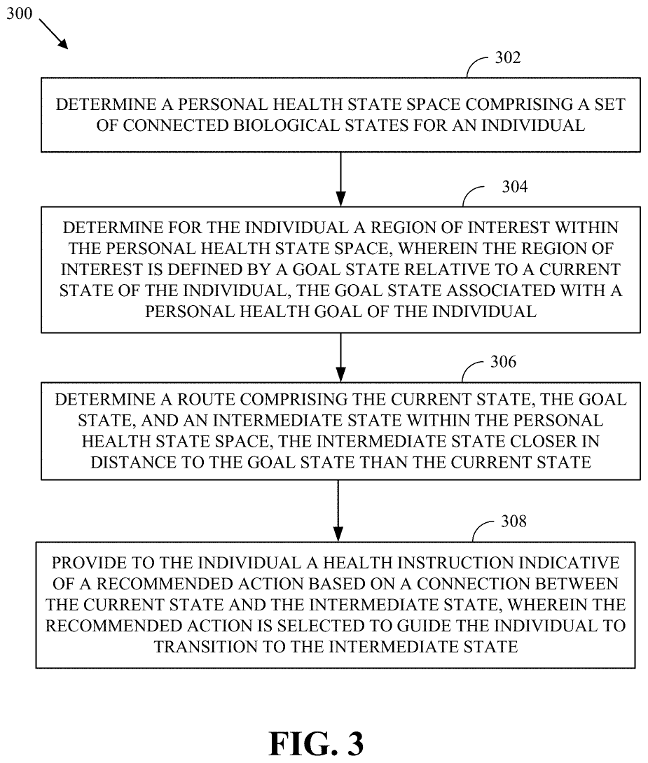

FIG. 3 is a diagram showing an example process 300 of personal health navigation according to implementations of this disclosure. In some implementations, some or all of the process 300 can be implemented in a device or apparatus such as the computing device 200 shown in FIG. 2. In some implementations, portions of the process 300 can be performed by instructions executable on the computing device 200 and/or one or more other devices, such as a wearable device or a mobile phone. In some implementations, the computing device 200 itself can be a mobile phone. In other implementations, the computing device 200 can be a wearable device, such as a smart watch. or a cloud server.

At an operation 302, a personal health state space (PHSS) is determined for an individual. The personal health state space includes a set of connected biological states for an individual. The biological states are also referred to herein as nodes or health states.

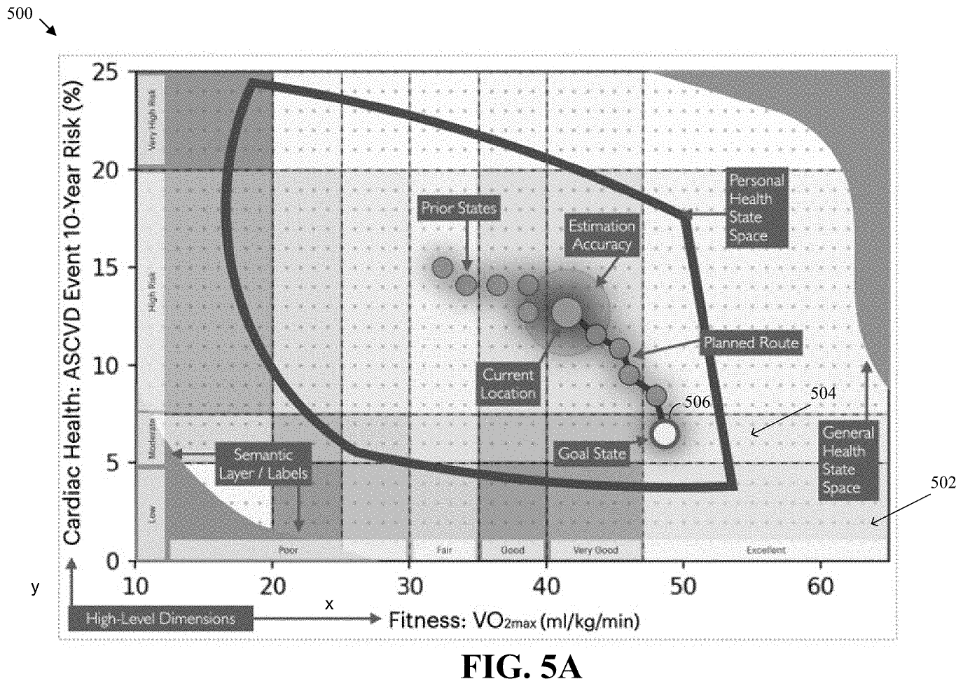

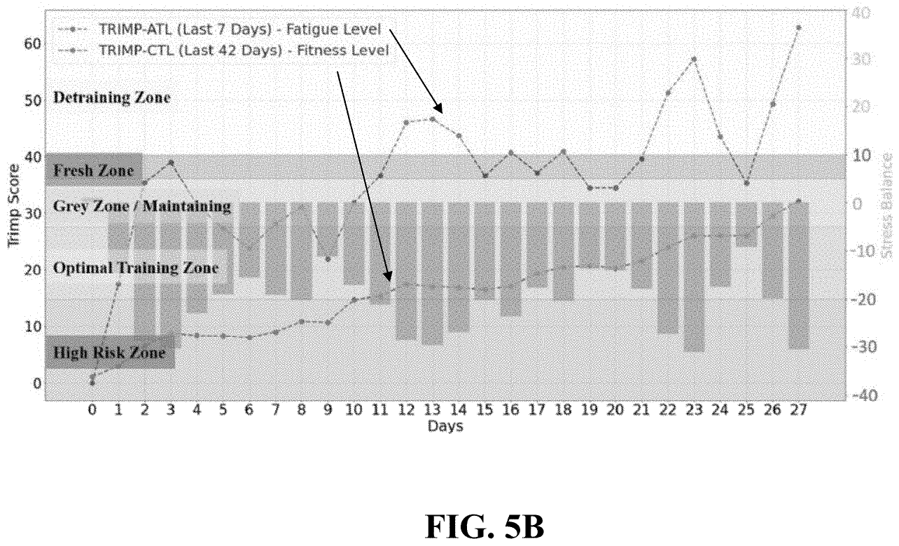

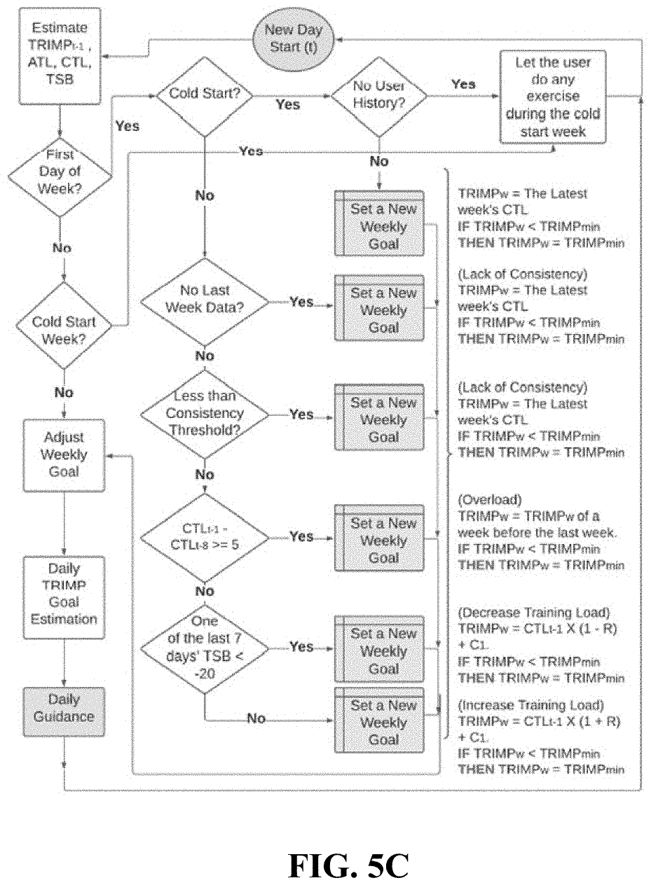

The personal health state space can be determined from, for example, a general health state space (GHSS). The PHSS is a subset of the general health state space. As discussed in FIG. 1, the general health state space includes all possible health states a human can exist in. For example, in a scenario of cardiovascular health, the GHSS includes all possible measurements of cardiovascular states for a human. The measurements can include, for example, heart rates (such as PPG, EGK or ECG measurements) or VO2max. The measurements can also include any components of fitness, cardiovascular diseases, and other medical events. The GHSS can be, for example, a multi-dimensional health state space having more than one type of measurements as components. An example of such a multi-dimensional health state space is discussed below in connection with FIGS. 5A-5C. Depending on a goal or domain(s) of interest specified by the individual, a corresponding set of dimensions relevant to the goal or domain of interest can be identified.

As discussed, when the GHSS is applied to an individual, the PHSS is determined, which includes a subset of possible biological states for the individual within the GHSS. In other words, the PHSS includes all the possibilities for the individual given the personal situation. The subset of possible biological states for the individual can include, for example, those biological states that are attainable by the individual, based on characteristics specific to the individual. For example, the characteristic specific to the individual used for generating the PHSS can include characteristics regarding at least one of genetics or demographics (e.g., gender, age) specific to the individual, which can be used to provide boundary thresholds for determining the PHSS within the GHSS.

Within the PHSS, connections, also referred to as edges, can be established between the individual states. Each edge within the PHSS represents a transition that take the individual from one state to another. An edge between a first state and a second state includes knowledge of those actionable inputs that can cause a state transition between the first and second states. For example, when the biological state includes a cardiovascular component (also referred to as “heart state”), it can be determined which actionable inputs will transition the heart state. The state transition takes into consideration various potential actionable inputs that will lead to the next state. For example, exercise, medicine, experiencing stress, or nutrition (e.g., having a high salt meal) may, alone or in combination, lead to state transition(s) in the PHSS. Connections within the PHSS for the individual between two nodes are also unique based on the individual. For example, for a person A and a person B to improve heart health parameters, or to grow one pound of muscle, the actions needed for A can be different than B.

In some implementations, a state transition network is determined for the PHSS based on a personal model associated with the individual. The state transition network includes edges, also referred to as connections in the descriptions above. For the state transition network, an edge is representative of a transition from a first state to a second state based on the personal model associated with the individual. Personal models may use various levels of specificity, such as, for example, grouping multiple individuals as a sub-population if not enough data is available on an individual basis. There can be multiple personal models associated with the individual. For example, a non-comprehensive set of personal models as layers for the individual can include, for example, base physiological knowledge, multi-model data streams, demographic patterns, clinical medical research, geographic information systems, etc.

As discussed, various personal models can be layered for the individual as a single user. For example, image models can include, for example, facial recognition, skin analysis, or medical imaging such as CT/X-Ray/MRI etc. Audio models can include, for example, voice analysis, speech recognition, or music entertainment models. Emotion and behavioral models may traverse multiple modalities to understand how the individual reacts psychologically to inputs virtually or in real-world settings. Personalized Geographical Information Systems (GIS) informs how the context and environment augment changes in the user for location-based enhancement of services. Cross-Modal models allow fusion of many different data types related to the individual such as, for example, omics/genetics data, wearable device physiologic streams, or medical records, etc. Combinations of these media types include, for example, language analysis, virtual interaction models, or video/AR/VR models. In an example, these personalized models can be integrated by a remotely AI proctored video/AR based physical interaction, where physiological and genetic data of the users is taken into account with real-time 3D analysis to give feedback or guidance to the user during a rehabilitation therapy, gaming experience, or fitness exercise.

Back to FIG. 3, the state transitions and personal models can be learned by various machine learning techniques. Additionally, modelling clustering using traces of health states and state transitions, along with machine learning models developed based on these clusters, can speed up the learning time with improved personalization.

Knowledge layers and region(s) of interest, which is described in detail below, can be identified within the PHSS relevant to the domain(s) of interest. Layers on top of the PHSS contains relevant health domain knowledge, similar to physical maps that use latitude, longitude, and altitude for description. Information layers such as roads, oceans, country borders, and satellite imagery allow for navigation within the space, depending on the context (e.g., driving requires roads and traffic layers). Knowledge layers represent the real world, in which humans can make sense of their states concerning their interests.

At an operation 304, a region of interest within the personal health state space is determined for the individual. The region of interest is defined by a goal state relative to a current state of the individual. The goal state is associated with a personal health goal of the individual.

The current state for the individual can be determined in various ways, including, for example, being estimated based on physiological measurements of the individual by a wearable device, manually input by the individual or imported from an existing physiological profile, etc. In order to assign an accurate location within the PHSS, the latest data from the individual can be used to predict the current state. The current state can be determined as, for example, a location on the PHSS, with an accuracy range. Different applications may require different levels of accuracy in order to provide services to the individuals as users.

For example, monitoring a cardiovascular health state is useful to both endurance athletes and heart disease patients. Estimation techniques can be used in many applications, but health applications will require increasingly deep biological knowledge layers to define and refine the estimated health states that are computed from incoming data.

The value of good health to an individual is largely based upon how they wish to live. For personal health navigation to occur, one or more personal health goals can be specified by the individual. A personal health goal can be specified by the individual, for example, to include one or more states in a region of interest (ROI). The ROI can also be specified as the personal health goal. Examples of ROI include the ROI 112 and the ROI 114 as discussed in FIG. 1, and more examples are to be discussed below in FIG. 5A.

The ROI is defined according to a domain of interest to the individual. Therefore, different ROIs or goals are associated with different domains of interest. The ROI can also be associated with a multi-dimensional space, where the dimensions represent different components of health as defined through biomedical knowledge.

Goal decomposition can be used to translate the goal state into (often short-term) sub-goals. This process includes identifying the unique utilities relevant for the specified goal.

The ROI can be semantically labeled with domain knowledge associated with the personal health goal. In some implementations, an input indicative of the personal health goal is received from the individual. The personal health goal is then decomposed into sub-goals represented as nodes in the ROI.

At an operation 306, a route is determined within the region of interest. The route includes, for example, the current state, the goal state, and an intermediate state, which includes one or more intermediate states within the region of interest. An intermediate state is closer in distance to the goal state than the current state.

In some implementations, for the individual associated with the personal health goal, the route leading from the current state to the goal state can be determined within the state transition network, and the route comprises an optimal subset of states between the current state and the goal state, and corresponding edges.

After measuring, estimating, modeling the individual, and receiving the personal health goal, the route to neighboring states that lead towards the goal state can be determined, so that the user receives guidance on the next step(s) needed to reach the personal health goal. The intermediate states and sub-goals between the current state and the goal state can be determined, along with the costs and constraints to transition between the intermediate states along the route. Route planning can include, for example, computing the best set of inputs to produce a state change to the neighboring state on the route towards the goal. For example, means-ends analysis and other techniques can be used with routing algorithms to determine the best intermediate states for the individual to reach the personal health goal. There can be multiple routes to reach a desired goal, and route selection can be based on one or more criteria such as, for example, user preferences, efficiency, speed, or available resources.

Having a map, location, and goal sets the stage for routing from the current state towards the goal state. The navigation allows a user to move forward through intermediate states over time towards a desired goal state. Making a route on a map requires not only knowing the start and endpoints but also all the layers of roads and traffic. In the case of PHN, each set of intermediate states and sub-goals will have its layer of information, which is relevant for mapping, along with costs and constraints to transition among intermediate states. Interactions in PHN can be extremely complex due to its large dimensionality. Competing user goals often need to be handled, through methods such as prioritization or weighting. Means—ends analysis or other problem-solving techniques, along with appropriate routing algorithms, can reveal the best intermediate states for the user to reach the goal. There may be multiple routes to get to the desired goal. However, route selection may be made by various optimization criteria that include, for example, at least one of user preferences, efficiency, speed, or resources.

After measuring, estimating, modeling the individual, and receiving a goal, the individual needs to receive guidance on the next step(s) needed to reach that goal. For the individual to make decisions about events leading up to the goal state, the PHN routes through intermediate steps through the PHSS. At each moment in time instructions are given for the next appropriate actions. Actionable inputs that can be part of the guidance include lifestyle events (exercise, nutrition, sleep, meditation, etc.) or medical events (medications, procedures, etc.), or a combination of the above.

At an operation 308, a health instruction indicative of a recommended action is provided to the individual. The recommended action is based on a connection between the current state and the intermediate state, and the recommended action is selected to guide the individual to transition to the intermediate state.

Cybernetic control is used to steer the individual to enact actions to transition the health state. State transition as a result of the control can be described in the following equations:

X[k+1]=A[k]X[k]+B[k]U[k] (Equation 1)

Y[k]=C[k]X[k]+D[k]U[k] (Equation 2)

where X, U, and Y are the system true state, inputs, and measured output vectors respectively. A, B, C, and D are matrices that provide the appropriate transformation of these variables at a given time k. An individual's health state at time k is denoted by X[k], and inputs (actions taken or to be taken) at time k is denoted by U[k], both of which play a role in determining the individual's health state at time k+1, denoted by X [k+1].

At each moment, such as k or k+1, instructions are given for the next appropriate actions. As discussed, actionable inputs that can be part of the guidance that include at least one of lifestyle events (e.g., exercise, nutrition, meditation, etc.) or medical events (e.g., medications, procedures, etc.). Therefore, the health instruction indicative of the recommended action can include, for example, at least one of a lifestyle event or a medical event. A key difference between navigation and recommendation is that recommendation for a particular point in time alone does not consider routing through the state space to the goal state.

Cybernetic control can also be obtained through automatic actuation of actions as inputs, such as changing the thermostat and lighting in the home and screen devices to automatically help re-adjust circadian rhythm from jetlag. Minimum data requirements (e.g., accuracy, sampling frequency etc.) should be considered in order to provide effective control in a given setting.

Actions carried out by the individual, including the recommended action or other actions, which can be measured, are used for determining a new estimation of the current state.

In some implementations, upon determining that the individual has completed the recommended action, the current state is transitioned into the intermediate state.

In some implementations, upon determining that the individual has taken a different action that transitions into another state different from the intermediate state, the route for the individual is recalculated based on the another state and the goal state.

Once the new estimation of the current state is performed, the process 300 can go back to the operation 306 for determining the new route to the goal state. Next, at the operation 308, an updated recommended action is determined and provided to the individual. A cycle including the operation 306 when the current state is updated and the operation 308 is performed repeatedly, until the individual reaches the goal state.

In some implementations, upon reaching the goal state, the process 300 can continue to calculate and send health instructions to minimize deviation from the goal state. This can be done by, for example, repeating the cycle of the operation 306 and the operation 308, as discussed above.

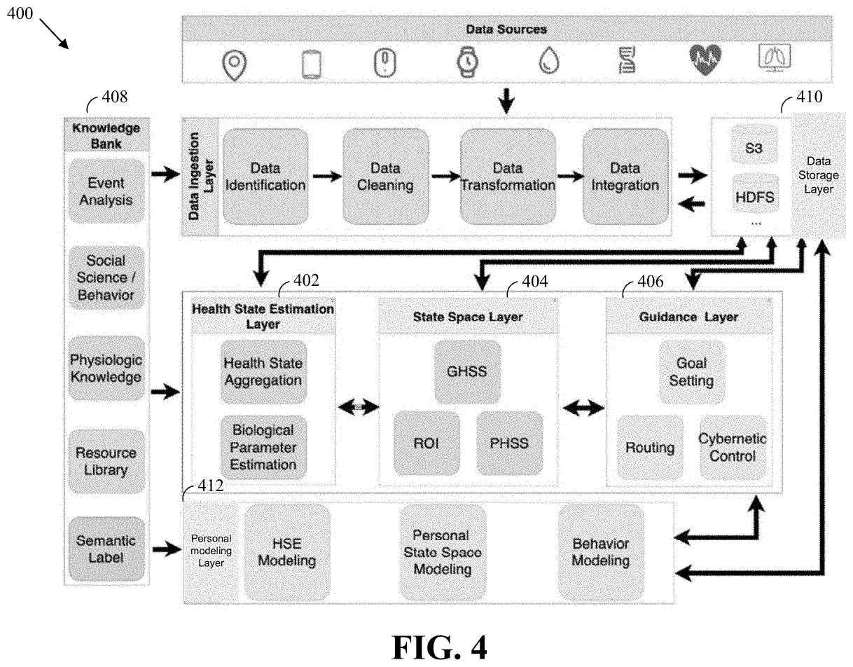

FIG. 4 is an example flow diagram illustrating an example system 400 framework for PHN. Broadly, the PHN functionality is divided into multiple layers including a Health State Estimation Layer 402, a State Space Layer 404, and a Guidance Layer 406, as shown in FIG. 4, which have been described above in connection with FIG. 1 and the process 300 of FIG. 3. Other layers such as Knowledge Bank 408, Data Storage Layer 410, Personal Modeling Layer 412, etc., can also be included. In some implementations, some or all of the system 400 can be implemented by a process such as the process 300 in FIG. 3 in a device or apparatus such as the computing device 200 shown in FIG. 2. In some implementations, portions of the system 400 can be performed by instructions executable on the computing device 200 and/or one or more other devices, such as a wearable device, a mobile phone or a cloud server. In some implementations, the computing device 200 can be a mobile phone, a wearable device, such as a smart watch. or a cloud server. Each layer as described below can be implemented in forms of a complete hardware implementation, a complete software implementation, or a combination of software and hardware implementation. The layers can be implemented as software modules, hardware, firmware, or a combination of the above.

Health State Estimation (HSE) Layer 402: It is important to note that there is constant flux within an individual's PHSS based on one's location in the state space. For success, navigation is required to assign an accurate location within the PHSS, just as GPS provides physical world navigation. The HSE layer 402 is used to determine this location, which requires ingesting the latest data from data storage layer 410 and domain knowledge from knowledge bank 408 to arrive at a predicted current state. For instance, in the case of a cardiovascular health state estimation, wearable sensor data, such as heart rate, activity, or step, can be obtained by the data storage layer 410, and estimating the cardiovascular disease (CVD), such as relative mortality risk using resting heart rate, VO2 Max, Power to Heart Rate Ratio, can be performed by the knowledge bank 408. The HSE layer can be used to determine a location with an accuracy range. The accuracy of the HSE is important to consider, as different applications require different levels of accuracy in order to provide services to the user. The same HSE tool may be useful for many applications. For example, monitoring a cardiovascular health state is useful to both endurance athletes and heart disease patients. Estimation techniques have been of great interest in designing many applications, but health applications will require an increasingly deep biological knowledge bank to define and refine the estimated health states that are computed from incoming data. Finally, the estimated health states can be shared with the state space layer 404 and stored in the data storage layer 410.

State Space Layer 404: The value of good health to an individual is largely based upon how they wish to live. When a goal specified by the individual is provided by the guidance layer 406, the system 400 can retrieve the appropriate state space based on the goal in the state space layer 404. This process includes identifying the unique set of dimensions, which can include health states estimated by the HSE layer 402 that are relevant for the specified goal. Goals can include states that can be specified as ROI(s) within these navigational dimensions. The GHSS describes the maximal size state space in which a human can exist. For example, when the state space of interest is the cardiovascular state, all possible estimations for the cardiovascular state can be considered, including all components of fitness, cardiovascular diseases, structural formation and more. This state space is then further refined for each individual into a PHSS based on individual characteristics (e.g., genetics, gender, age) specific to the individual that provide boundary thresholds. This PHSS infused by ROI(s) is shared with the guidance layer 406, HSE layer 402, and personal modeling layer 412.

Personal Modeling Layer 412: Within the PHSS, there are connections/edges made between each of the individual state possibilities, as shown in the example in FIG. 1. Each edge within the network represents transitions that take the person from one node to another. The state transition takes into consideration all the inputs (actions) that will lead to the next state, therefore making connections or relationships between states. Personal modeling transforms the actional inputs to the predicted output. In the personal modeling layer 412, the system 400 discovers these relationships in the data and predicts how a certain input should affect the current health state by extending the model into a future time point. The personal model can include various types of relationship mapping. The HSE modeling is used to understand precisely how specific inputs cause an effect on the health state of an individual. Inputs can vary from lifestyle choices, medicine, environment, and more. Biologically, the inputs cause a change in metabolism and gene expression in the user's cells which then change the structure and function of tissue in the organs. This change of biological architecture is reflected in a changed health state. The personal state space modeling can be used to assist the PHSS in more detail. It requires identifying the knowledge layers and ROI within the space relevant to the topic of interest. these relationships are mapped with known domain knowledge in the personal modeling layer 412. The domain knowledge is transferred to a rule-based algorithm that can represents current understanding of biomedical sciences. Once each individual user accumulates enough data, the personal model can be refined by modifying the base personal model by matching the user's patterns to data-driven clustering of user sub-groups. The subgroup can be then modified for the individual with data produced only through the individual. Personal models may use various levels of specificity, such as grouping as a sub-population, as is common in cold start scenarios. Additionally, modelling clustering using traces of health states and state transitions and developing machine learning models based on these clusters may lead to improved personalization while speeding-up the learning time. As discussed above in connection with FIG. 3, the state transition network can also be included as a unique layer on top of the PHSS.

Guidance Layer 406: After measuring, estimating, modeling the individual, and receiving a goal, guidance is provided on the next step the individual needed to accomplish to reach that goal. Having a map, location, and goal in the guidance layer 406 sets the stage for routing from the current state towards the goal state as shown in the example in FIG. 1. Making a route on a map requires not only knowing the start and endpoints but also connecting the two. PHN computes the set of intermediate states and sub-goals with its layer of information, which is relevant for mapping, along with costs and constraints to transition between intermediate states along the planned route. Problem solving techniques, along with appropriate routing algorithms, can be used to determine the best intermediate states for the user to reach the goal. There may be multiple routes to get to the desired goal. However, route selection could be made by various optimization criteria that include user preferences, efficiency, speed, and available resources. Control mechanisms steer individuals towards the state transition on various timescales. At each moment in time, instructions are given for the next appropriate actions. Actionable inputs that can be part of the guidance include at least one of lifestyle events (exercise, nutrition, sleep, meditation, etc.) or medical events (medications, procedures, etc.).

FIG. 5A is an example diagram 500 illustrating applying PHN in a cardiovascular health setting. According to research, cardiovascular health is the most significant cause of death in humans. Clinical need for improving cardiovascular health and cardio-respiratory fitness (CRF) is in high demand, but often addressed only in high need care or critical situations using expensive lab tests and rehabilitation programs. By using lower cost wearable devices employing PHN, this task can be accomplished.

According to an implementation in this use case, PHN is deployed to improve cardiovascular health and cardio-respiratory fitness (CRF) of each individual. Other scenarios, such as improving mental health, athletic training for a particular event (e.g., marathon), can also utilize the PHN. Multiple goals can also be combined. Examples of application scenarios can include, for example, food, exercise, sleep, shopping, travel, business etc.

Sensor data streams are received from wearable device (e.g., smart watch, wristband, or earbud) and/or mobile device of the individual, which can be aggregated. The sensor data streams can include, for example, at least one of time stamp, step count, HR (BPM), activity mode (e.g., still, walking, running), sleep (e.g., deep sleep, light sleep, rem sleep, sleep score), resting HR, age, gender, height, weight, or the like. Domain knowledge for building the PHSS can include, for example, knowledge about at least one of sport science, or bioenergetics science. Medical data can include, for example, at least one of ASCVD or cardiac risk factors. ASCVD refers to Atherosclerotic Cardiovascular Disease (ASCVD), which is measured by cholesterol levels, diabetes status, smoking habits, blood pressures, age, and gender.

To estimate the cardiovascular health state, cardiac disease risk can be determined by weighting the individual's ASCVD risk, which can be further enhanced with a relative risk modifier of resting heart rate extracted during deep sleep. Deep sleep can be determined by, for example, episodic medical blood data with high frequency sensing of a wearable device. Indicators of maximum oxygen consumption capacity (VO2Max) can be determined from exercises, such as, for example, steps or walking.

According to FIG. 5A, two dimensions, ASCVD and VO2Max, denoted by y-axis and x-axis respectively, are used to generate a GHSS heart map. The GHSS can be determined, for example, according to the descriptions in FIGS. 1, 3 and 4 (e.g., the GHSS 101, process 302, etc.).

The domain knowledge about cardiovascular health is then applied to the GHSS. The PHSS can be determined from the GHSS using the demographic information (e.g., age, gender) of the individual. The PHSS can be determined, for example, according to the descriptions in FIGS. 1, 3 and 4 (e.g., the PHSS 102, the operation 302, etc.). Also, the ROI(s) can be determined. For example, a ROI 504 indicates a ASCVD risk of “moderate” and VO2Max of “Excellent”. Another ROI 502 indicates a ASCVD risk of “low” and VO2Max of “Excellent”. A goal state 506, in this example, falls into ROI 504.

For example, a rule-based HSE model can be built with domain knowledge derived from bioenergetics science. The HSE model can be determined, for example, according to the descriptions in FIGS. 1, 3 and 4 (e.g., the HSE 104, or the operation 304). With this model, an actionable daily exercise guidance through the cardiac PHN system can be sent to participants and the changes in the individual CRF indicators are monitored.

To build the personal module for the cardiac PHN system, the knowledge layer about how increasing intensity and duration of exercise lowers risk of cardiovascular disease is used. Advanced physiologic cardiovascular endurance training strategies in bioenergetics science can also be used to build a personalized rule-based model for daily exercise guidance. Table I below explains the definitions used in the rule-based model. One or more of the following rules can be used in the guidance module:
TSB≥+10: Transition Zone. User is well rested. The value is often reached when a user has an extended break.
+5≤TSB<+10: Fresh Zone. Zone reached when user is optimally recovered from exercise.
−5≤TSB<+5: Neutral Zone. Zone reached typically when a user is in rest or recovery week.
−30≤TSB<−5: Optimal Training Zone. Zone where the user can best build their effective fitness.

−30≥TSB: Over Load Zone. User is over-training and should take a rest to protect from injury.

TSB should be maintained in the optimal training zone to improve ASCVD and VO2Max.

TRIMP_{w}=CT Lₜ₋₁×(1+R)+C₁ (Equation 3)
CTL increase with a maximal rate limit not exceeding 5 per week.

CT Lₜ₋₁−CT Lₜ₋₈<5 (Equation 4)
TSB shouldn't drop below −20 more than once in 10 days.
If the TSB dropped below −20 in a given week, the next week's training goal should be scaled down slightly.

TABLE I

| Term | Description |
|---|---|
| TRIMP | (TRaining IMPulse) Weighted product of |
| | training volume and training intensity by HR. |
| CTL | Chronic Training Load (Fitness Level). |
| | Average TRIMP score for the recent 6 weeks. |
| ATL | Acute Training Load (Fatigue Level). |
| | Aver-age TRIMP score for the recent 1 week. |
| TSB | Training Stress Balance. CTL - ATL. |
| TRIMPₘᵢₙ | Minimum Weekly Goal based on CDC guidance. |
| TRIMP_{d} | Daily TRIMP goal |
| TRIMP_{w} | Weekly TRIMP goal |
| R | Ramp Rate (0 ≤ R ≤ 1) |
| C₁ | Optimal Training Zone Coefficient (5 ≤ C1 ≤ 30) |
| t | Current day |

With the rule-based model defined in the personal modeling layer, a guidance module can be built to include routing and control. With the rules in the guidance module, the proper intensity and duration of exercise can be gradually determined over time with the cybernetic control in the PHN system. Additionally, training stress balance (TSB), which reflect each participant's current fatigue level and fitness level, can be used to show the current states of the users and provide the adjusted guidance in the cybernetic loop, even in cases that the participants did not always follow the PHN guidance.

FIG. 5B is an example diagram illustrating user trends of fitness level, fatigue level, and stress balance for PHN according to experimental data collected from the cardiovascular health setting. FIG. 5B shows that the PHN system is gradually improving the individual's fatigue level (ATL) and fitness level (CTL) while maintaining TSB in the “Optimal Training Zone.” The individual user is able to monitor and keep lifestyle on track by keeping the bars within the “Optimal Training Zone.”

FIG. 5C shows an example flow diagram of the daily exercise guidance algorithm. By using this guidance algorithm, an individual can be guided to reach and maintain within their optimal training zone as shown in FIG. 5B, which can potentially help improve the levels of CRF. A daily exercise guidance can include, for example, exercise type, exercise intensity, and exercise duration, such as, for example, “Go jogging for at least 40 minutes sustaining HR above 113 bpm.” A daily TRIMP goal can be converted into a recommendation action set as follows:

$$•Low - intensity workout for \frac{{TRIMP}_{d}}{{c}_{1}} minutes while sustaining 0.55 \times Max HR ⇐ HR < 0.7 \times Max HR .$$

$$•Medium - intensity workout for \frac{{TRIMP}_{d}}{{c}_{2}} minutes while sustaining 0.7 \times Max HR ⇐ HR < 0.8 \times Max HR .$$

$$•High - intensity workout for \frac{{TRIMP}_{d}}{{c}_{3}} minutes while sustaining 0.8 \times Max HR ⇐ HR ⇐ 1. \times Max HR .$$

where c1, c2, and c3 are the Lucia's coefficients, which can be 1, 2, and 3, respectively.

According to another example in the scenario of cardiovascular health setting, a personal model on cardiac exercise response is used. According to this example, the personal model shows that a standard dose of exercise would not effectively impact each participant's cardiovascular health state in the same way. Therefore, connections within a PHSS between nodes are shown to be unique for the individual.

The exercise groups of individuals can be defined based on, for example, frequency (e.g., Low: 1 time per week, Mid: 2-4 times per week, High: 5-7 times per week), amount (e.g., Low: less than 30 minutes per exercise, High: more than 30 minutes per exercise), and intensity of exercise (e.g., Low: lower than 75% of estimated maximal heart rate, High: greater than 75% of estimated maximal heart rate). Each individual can be categorized into a corresponding exercise group based on the above criteria. For example, an individual can belong to the following group: High (Frequency)-High (Amount)-High (Intensity).

Strong causal relationships connect CRF to predicting cardiovascular disease. Additionally, epidemiological research shows that resting heart rate is an independent predictor of cardiovascular disease. Therefore, the resting heart rate can be used as an indicator of CRF, and each individual's cardiovascular exercise response can be labeled as, for example, positive (Vᵣₕᵣ(t)<−0.5), neutral (−0.5<=Vᵣₕᵣ(t)<=0.5), and negative responder (Vᵣₕᵣ(t)>0.5) by the velocity of resting HR change:

$${V}_{rhr} ( t ) = \frac{{\sum }_{i = 1}^{t} ( {x}_{i} - \overline{X} ) ( {y}_{i} - \overline{Y} )}{{\sum }_{i = 1}^{t} {( {x}_{i} - \overline{X} )}^{2}} , \qquad ( Equation 5 )$$

where t is total weeks, xᵢ is 1, 2, 3, . . . , t, yᵢ is the resting heart rate of iₜₕ week, X̅ is the sum of xᵢ, and Y̅ is the sum of yᵢ. A positive responder means a person whose resting heart rate decreased after certain weeks of exercise. A negative responder means a person whose resting heart rate increased after certain weeks of exercise. A neutral responder means a person who didn't have a big change in his resting heart rate even after certain weeks of exercise.

Building a personal model can benefit from finding inter-individual differences over time and personalizing the model with these factors, such as with federated learning. A majority of the people (e.g., 67% according to our experiments) may not show standard responsiveness to external influences. Since it is difficult to know these differences initially, a sub-population based model can be used, if there is not enough data readily available from a single user, followed by incrementally reinforcing the personal model in a closed-loop cycle of the PHN framework.

Individual user's exercise, heart rate and sleep data are processed and analyzed. Experimental data shows there are clustering in CRF responses from different individuals. Cluster information matched to a user CRF response type can then be applied to provide personalize daily guidance for the individuals. Additionally, individual data can be used to model personal response lag time for predicting state transition in the PHSS.

Additionally, in some implementations, PHN can be used, for example, in the following scenarios:

Early Detection and Preventive Action: Insight into health status changes in the prodromal state, where health state can be readily shifted towards wellness and disease can be avoided.

Continuous Weaving of User Data for Various Applications: Understanding data to assisting the individual in all aspects of life such as sports, entertainment, shopping, travel, enjoying better food, and improving quality of life.

Quantitative Interactivity of Health: Shifting health assessment to a dynamic quantitative measurement rather than categories of normal versus abnormal, which empowers individuals to participate in their health.

Reduced Cost: Through trickle down technology and scale of computing, we anticipate more people will have access to high quality guidance, reducing cost barriers to living healthy, especially in developing areas without developed physical healthcare infrastructure.

Technical specialists skilled in the art should understand that, the implementations in this disclosure may be implemented as methods, systems, or computer program products. Therefore, this disclosure may be implemented in forms of a complete hardware implementation, a complete software implementation, and a combination of software and hardware implementation. Further, this disclosure may be embodied as a form of one or more computer program products which are embodied as computer executable program codes in computer writable storage media (including but not limited to disk storage and optical storage).

This disclosure is described in accordance with the methods, devices (systems), and flowcharts and/or block diagrams of computer program products of the implementations, which should be comprehended as each flow and/or block of the flowcharts and/or block diagrams implemented by computer program instructions, and the combinations of flows and/or blocks in the flowcharts and/or block diagrams. The computer program instructions therein may be provided to generic computers, special-purpose computers, embedded computers or other processors of programmable data processing devices to produce a machine, wherein the instructions executed by the computers or the other processors of programmable data processing devices produce an apparatus for implementing the functions designated by one or more flows in the flowcharts and/or one or more blocks in the block diagrams.

The computer program instructions may be also stored in a computer readable storage which is able to boot a computer or other programmable data processing device to a specific work mode, wherein the instructions stored in the computer readable storage produce a manufactured product containing the instruction devices which implements the functions designated by one or more flows in the flowcharts and/or one or more blocks in the block diagrams.

The computer program instructions may also be loaded to a computer or another programmable data processing device to execute a series of operating procedures in the computer or the other programmable data processing device to produce a process implemented by the computer, whereby the computer program instructions executed in the computer or the other programmable data processing device provide the operating procedures for the functions designated by one or more flows in the flowcharts and/or one or more blocks in the block diagrams.

Apparently, the technical specialists skilled in the art may perform any variation and/or modification to this disclosure by the principles and within the scope of this disclosure. Therefore, if the variations and modifications herein are within the scope of the claims and other equivalent techniques herein, this disclosure intends to include the variations and modifications thereof.

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. The terms “at least one of A or B,” “at least one of A and B,” “one or more of A or B,” “A and/or B” used herein mean “A”, or “B” or “A and B”.

While the disclosure has been described in connection with certain embodiments or implementations, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for personal health navigation, comprising:
determining, by a processor, a personal health state space comprising a set of connected biological states for an individual;
determining, for the individual and by the processor, a region of interest within the personal health state space, wherein the region of interest is defined by a goal state relative to a current state of the individual, the goal state associated with a personal health goal of the individual;
determining, by the processor, a route comprising the current state, the goal state, and an intermediate state within the personal health state space, the intermediate state closer in distance to the goal state than the current state; and
providing, to the individual and by the processor, a health instruction indicative of a recommended action based on a connection between the current state and the intermediate state, wherein the recommended action is selected to guide the individual to transition to the intermediate state.

2. The method of claim 1, wherein upon determining that the individual has completed the recommended action, the current state is transitioned into the intermediate state.

3. The method of claim 1, further comprising:
upon determining that the individual has taken a different action wherein the current state is transitioned into another state different from the intermediate state, recalculating the route for the individual based on the another state and the goal state.

4. The method of claim 1, wherein the region of interest is semantically labeled with domain knowledge associated with the personal health goal.

5. The method of claim 1, further comprising:
receiving, from the individual, input indicative of the personal health goal; and
decomposing, by the processor, the personal health goal into sub-goals represented as nodes in the region of interest.

6. The method of claim 1, further comprising:
determining, by the processor, a state transition network comprising edges, each edge representative of a transition from a first state to a second state based on a personal model associated with the individual.

7. The method of claim 6, wherein determining, by the processor, the route comprising the current state, the goal state, and the intermediate state further comprises:
determining, for the individual associated with the personal health goal, the route leading from the current state to the goal state within the state transition network, wherein the route comprises an optimal subset of states between the current state and the goal state, and corresponding edges.

8. The method of claim 1, wherein the health instruction indicative of the recommended action comprises one of: a lifestyle event or a medical event.

9. The method of claim 1, wherein the current state is estimated based on physiological measurements of the individual by a wearable device.

10. The method of claim 1, wherein the personal health state space comprises a subset of possible biological states for the individual within a multi-dimensional general health state space, the subset of possible biological states determined based on characteristics specific to the individual.

11. An apparatus for personal health navigation, the apparatus comprising:
a non-transitory memory; and
a processor configured to execute instructions stored in the non-transitory memory to:
determine a personal health state space comprising a set of connected biological states for an individual;
determine, for the individual, a region of interest within the personal health state space, wherein the region of interest is defined by a goal state relative to a current state of the individual, the goal state associated with a personal health goal of the individual;
determine, by the processor, a route comprising the current state, the goal state, and an intermediate state within the personal health state space, the intermediate state closer in distance to the goal state than the current state; and
provide, to the individual and by the processor, a health instruction indicative of a recommended action based on a connection between the current state and the intermediate state, wherein the recommended action is selected to guide the individual to transition to the intermediate state.

12. The apparatus of claim 11, wherein upon determining that the individual has completed the recommended action, the current state is transitioned into the intermediate state.

13. The apparatus of claim 11, further comprising:
upon determining that the individual has taken a different action wherein the current state is transitioned into another state different from the intermediate state, recalculating the route for the individual based on the another state and the goal state.

14. The apparatus of claim 11, wherein the region of interest is semantically labeled with domain knowledge associated with the personal health goal.

15. The apparatus of claim 11, wherein the instructions further comprise instructions to:
receive, from the individual, input indicative of the personal health goal; and
decompose the personal health goal into sub-goals represented as nodes in the region of interest.

16. The apparatus of claim 11, wherein the instructions further comprise instructions to:
determine a state transition network comprising edges, each edge representative of a transition from a first state to a second state based on a personal model associated with the individual.

17. The apparatus of claim 16, wherein the instructions to determine the route comprising the current state, the goal state, and the intermediate state within the region of interest further comprise instructions to:
determine, for the individual associated with the personal health goal, the route leading from the current state to the goal state within the state transition network, wherein the route comprises an optimal subset of states between the current state and the goal state, and corresponding edges.

18. The apparatus of claim 11, wherein the health instruction indicative of the recommended action comprises one of: a lifestyle event or a medical event, and wherein the current state is estimated based on physiological measurements of the individual.

19. The apparatus of claim 11, wherein the personal health state space comprises a subset of possible biological states for the individual within a multi-dimensional general health state space, the subset of possible biological states determined based on characteristics specific to the individual.

20. A non-transitory computer-readable storage medium configured to store computer programs for personal health navigation, the computer programs comprising instructions executable by a processor to:
determine a personal health state space comprising a set of connected biological states for an individual;
determine, for the individual, a region of interest within the personal health state space, wherein the region of interest is defined by a goal state relative to a current state of the individual, the goal state associated with a personal health goal of the individual;
determine, by the processor, a route comprising the current state, the goal state, and an intermediate state within the personal health state space, the intermediate state closer in distance to the goal state than the current state; and
provide, to the individual and by the processor, a health instruction indicative of a recommended action based on a connection between the current state and the intermediate state, wherein the recommended action is selected to guide the individual to transition to the intermediate state.
